(19) United States
(12) Patent Application Publication
(10) Pub. No.: US 2025/0235711 A1
(43) Pub. Date: Jul. 24, 2025

(54) ELECTROMAGNETIC WAVE DEVICE AND METHOD FOR USING THE SAME

(71) Applicants: Jongwoon Choi, Seoul (KR); DSE Inc., Seoul (KR)

(72) Inventor: Jongwoon Choi, Seoul (KR)

(21) Appl. No.: 18/985,717

(22) Filed: Dec. 18, 2024

(30) Foreign Application Priority Data
Jan. 24, 2024 (KR) 10-2024-0010781

(51) Int. Cl. A61N 5/00

(52) U.S. Cl. CPC A61N 5/00

(57) ABSTRACT

The present disclosure relates to an electromagnetic wave therapy device and an electromagnetic wave treatment method using the same. The electromagnetic wave therapy device includes: an electromagnetic wave generator generating a traveling wave; an emitter receiving the traveling wave and emitting a reflected wave; a coupler connecting the electromagnetic wave generator and the emitter and measuring magnitudes of the traveling wave and the reflected wave; a comparer/determiner operating the electromagnetic wave generator when a ratio of the measured traveling wave and reflected wave reaches a preset value; and a controller operating the electromagnetic wave generator only when the emitter is in contact with the skin by controlling the electromagnetic wave generator, the emitter, the coupler, and the comparer/determiner, or a capacitive touch sensor, whereby generating a traveling wave only when the emitter is in contact with a skin.

### CROSS-REFERENCE TO RELATED APPLICATION

This application claims the benefit of priority under 35 U.S.C. § 119 (a) to Korean Patent Application No. 10-2024-0010781, filed on Jan. 24, 2024, the disclosure of which is incorporated herein by reference in its entirety.

### BACKGROUND

### (a) Technical Field

The present disclosure relates to an electromagnetic wave therapy device and a treatment method using the same, and more specifically to an electromagnetic wave therapy device that generates electromagnetic waves only when it is in contact with the skin, and a treatment method using the same.

### (b) Background Art

Recently, in the current trend of increasing interest in skin beauty and skin health, there is growing attention to electromagnetic wave therapy devices that restore skin elasticity, as more people around the world are seeking skin procedures.

In particular, through procedures using electromagnetic wave therapy devices, RF electromagnetic waves or microwaves are used to stimulate collagen and elastin fibers in the skin, whereby it is possible to improve sagging skin and increase elasticity, and the recovery time after treatment and procedures is short, so many people prefer procedures using electromagnetic wave therapy devices.

When therapeutic radiofrequency waves are irradiated to vulnerable areas of the body, such as the eyes, reproductive organs, and head, the electromagnetic waves may have harmful effects on the body. Accordingly, efforts are being made to develop electromagnetic wave therapy devices that are safer, more efficient, and capable of delivering effective electromagnetic wave treatments and procedures.

However, despite various attempts and significant progress made in the development of high-performance and effective electromagnetic wave therapy devices, there is a problem that there are no established standards for safely administering treatments and procedures on humans and it is difficult to control absorption of electromagnetic waves by the body.

Therefore, there is a need to develop an electromagnetic wave therapy device that minimizes absorption of electromagnetic waves by the body for purposes other than the intended therapeutic purpose in electromagnetic wave treatments and procedures.

### PRIOR ART DOCUMENT

### Patent Document

European Patent No. 3422978

### SUMMARY OF THE DISCLOSURE

It is an object of the present disclosure to solve the problems described above and other problems. It is another object to provide an electromagnetic wave therapy device and a treatment method using the same.

In order to achieve the above-mentioned or other objects, according an aspect of the present disclosure, there may be provided an electromagnetic wave therapy device that treats a skin by generating an electromagnetic wave at an end that is in contact with a skin, the electromagnetic wave therapy device including: an electromagnetic wave generator generating a traveling wave; an emitter receiving the traveling wave and emitting a reflected wave; a coupler connecting the electromagnetic wave generator and the emitter and measuring magnitudes of the traveling wave and the reflected wave; a comparer/determiner operating the electromagnetic wave generator when a ratio of the measured traveling wave and reflected wave reaches a preset value; and a controller operating the electromagnetic wave generator only when the emitter is in contact with the skin by controlling the electromagnetic wave generator, the emitter, the coupler, and the comparer/determiner.

According to an aspect of the present disclosure, when the ratio of the traveling wave and the reflected wave is smaller than the preset value, the electromagnetic wave generator may output a traveling pulse sufficient for detection of a traveling wave and a reflected wave and the comparer/determiner may measure a ratio of the traveling pulse and a reflected pulse reflected by the emitter; and when the ratio of the traveling pulse and the reflected pulse reaches a preset value, the electromagnetic wave generator may be operated.

According to an aspect of the present disclosure, the traveling pulse may be a pulse wave or a low-power electromagnetic wave that has electromagnetic wave energy sufficient for detection of the traveling pulse and the reflected pulse.

According to an aspect of the present disclosure, there may be provided an electromagnetic wave therapy device that treats a skin by generating an electromagnetic wave at an end that is in contact with a skin, the electromagnetic wave therapy device including: an electromagnetic wave generator generating a traveling wave; an emitter receiving the traveling wave and emitting a reflected wave; a capacitive touch sensor disposed between the electromagnetic wave generator and the emitter and measuring capacitance between the skin and the emitter; a comparer/determiner operating the electromagnetic wave generator when the value of the capacitance measured by the capacitive touch sensor reaches a preset value, and stopping the electromagnetic wave generator when the value of the capacitance is smaller than the preset value; and a controller operating the electromagnetic wave generator only when the emitter is in contact with the skin by controlling the electromagnetic wave generator, the emitter, the capacitive touch sensor, and the comparer/determiner.

According to an aspect of the present disclosure, there may be provided an electromagnetic wave treatment method using an electromagnetic wave therapy device that includes an electromagnetic wave generator generating a traveling wave, an emitter receiving the traveling wave and emitting a reflected wave, a coupler connecting the electromagnetic wave generator and the emitter and measuring magnitudes of the traveling wave and the reflected wave output from the electromagnetic wave generator, a comparer/determiner operating the electromagnetic wave generator when a ratio of the measured traveling wave and reflected wave reaches a preset value, and a controller operating the electromagnetic wave generator only when the emitter is in contact with the skin by controlling the electromagnetic wave generator, the emitter, the coupler, and the comparer/determiner, the electromagnetic wave treatment method including: (a) operating the electromagnetic wave generator; (b) comparing magnitudes of the traveling wave and the reflected wave; and (c) determining whether a ratio of the traveling wave and the reflected wave reaches a preset value, in which when the ratio of the traveling wave and the reflected wave is larger than the preset value in the determining, the electromagnetic wave generator is operated such that the electromagnetic wave generator is operated only when the emitter is in contact with the skin.

According to an aspect of the present disclosure, the electromagnetic wave treatment method may further include: when the ratio of the traveling wave and the reflected wave is smaller than the preset value in the comparing of the magnitudes of the traveling wave and the reflected wave, (d) stopping output of the traveling wave and generating a traveling pulse from the electromagnetic wave generator; (e) comparing magnitudes of the traveling pulse and a reflected pulse from the emitter; and (f) performing the step (a) when a ratio of the traveling pulse and the reflected pulse is larger than a preset value, and performing the step (d) when the ratio of the traveling pulse and the reflected pulse is smaller than the preset value.

According to an aspect of the present disclosure, there may be provided an electromagnetic wave treatment method using an electromagnetic wave therapy device that includes an electromagnetic wave generator generating a traveling wave, an emitter receiving the traveling wave and emitting a reflected wave, a coupler connecting the electromagnetic wave generator and the emitter and measuring magnitudes of the traveling wave and the reflected wave, a capacitive touch sensor connected at both ends to the emitter and the electromagnetic wave generator, respectively, and detecting capacitance when the emitter is in contact with a skin, a comparer/determiner operating the electromagnetic wave generator when capacitance detected by the capacitive touch sensor is larger than a preset value, and a controller operating the electromagnetic wave generator only when the emitter is in contact with the skin by controlling the electromagnetic wave generator, the emitter, the coupler, the capacitive touch sensor, and the comparer/determiner, the electromagnetic wave treatment method including: (g) operating the capacitive touch sensor; (h) comparing capacitance detected by the capacitive touch sensor and a preset value by means of the comparer/determiner; and (i) operating the electromagnetic wave generator when the detected capacitance is larger than the preset value.

According to an aspect of the present disclosure, when the detected capacitance is smaller than the preset value in the step (h), output of the traveling wave from the electromagnetic wave generator may be stopped and the step (h) may be performed.

An electromagnetic wave therapy device according to the present disclosure and a treatment method using the same are as follows.

According to at least one of embodiments of the present disclosure, it is possible to prevent electromagnetic waves from being emitted to a human body that is sensitive and vulnerable by preventing the situation that electromagnetic waves generated from the electromagnetic wave therapy device leaks to a free space. In particular, it is possible to prevent electromagnetic waves from being emitted to vulnerable areas of the body such as the eyes, reproductive organs, and head.

According to at least one of embodiments of the present disclosure, it is possible to solve the inconvenience of frequently turning on/off the electromagnetic wave therapy device during treatments and procedures by continuously checking whether the emitter comes in contact with a skin.

Further scope of applicability of the present disclosure will be made clear from the following detailed description. However, various changes and modification within the spirit and scope of the present disclosure can be clearly understood by those skilled in the art, so the detailed description and specific embodiments such as preferred embodiments of the present disclosure should be understood only as examples.

### BRIEF DESCRIPTION OF DRAWINGS

FIG. 1 is a configuration diagram illustrating an electromagnetic wave therapy device related to a first embodiment of the present disclosure.

FIG. 2 is a block diagram of an electromagnetic wave therapy device according to an embodiment of the present disclosure.

FIG. 3 is a configuration diagram illustrating an electromagnetic wave therapy device including a capacitive couch sensor related to a second embodiment of the present disclosure.

FIG. 4 is a flowchart of a treatment method using the electromagnetic wave therapy device related to the first embodiment.

FIG. 5 is a flowchart of a treatment method using the electromagnetic wave therapy device related to the second embodiment.

### DETAILED DESCRIPTION

Hereinafter, embodiments of the present disclosure will be described in detail with reference to the accompanying drawings and the same or similar components are given the same reference numerals regardless of the numbers of figures and are not repeatedly described. The term “unit” that is used for components in the following description is used only for the convenience of description without having discriminate meanings or functions. In the following description, if it is determined that the detailed description of known technologies related to the present disclosure makes the subject matter of the embodiments described herein unclear, the detailed description is omitted. Further, the accompanying drawings are provided only for easy understanding the embodiments described herein without limiting the spirit described herein and should be understood as including all of changes, equivalents, and substitutes included in the spirit and technical scope of the present disclosure. Singular forms include plural forms unless the context clearly indicates otherwise.

It will be further understood that the term “comprise” or “have” used in this specification specifies the presence of stated features, numerals, steps, operations, components, parts, or a combination thereof, but does not preclude the presence or addition of one or more other features, numerals, steps, operations, components, parts, or a combination thereof.

An electromagnetic wave according to an embodiment of the present disclosure refers to an electromagnetic wave, and more specifically is a concept that encompasses both a traveling wave radiated from a device generating electromagnetic waves to the surroundings and a reflected wave that is generated at a free-space incident surface duet o the significant impedance difference between an emitter 120 and the free space.

Further, an electromagnetic wave therapy device 100 according to an embodiment of the present disclosure refers to a device that treats the epidermis, fat layer, and muscle layer by positioning an emitter 120, which emits electromagnetic waves, close to a skin and transmitting electromagnetic waves into a human body. In more detail, an electromagnetic wave therapy device 100 that prevents electromagnetic waves from being emitted when the emitter 120 that radiates a traveling wave 20 is not in contact with the skin of human, and a treatment method using the electromagnetic wave therapy device 100 are disclosed.

Meanwhile, when an electromagnetic wave is emitted from the emitter 120 to a free space, a reflected wave 30 is generated from the emitter 120 due to the impedance difference between the emitter 120 and the free space. When the emitter 120 is in contact with a skin, most traveling waves 20 are absorbed into the skin, so the reflected wave 30 generated from the emitter 120 is minimized. Accordingly, it is possible to determine whether the emitter 120 is in contact with a skin by comparing the magnitudes of the traveling wave 20 and the reflected wave 30.

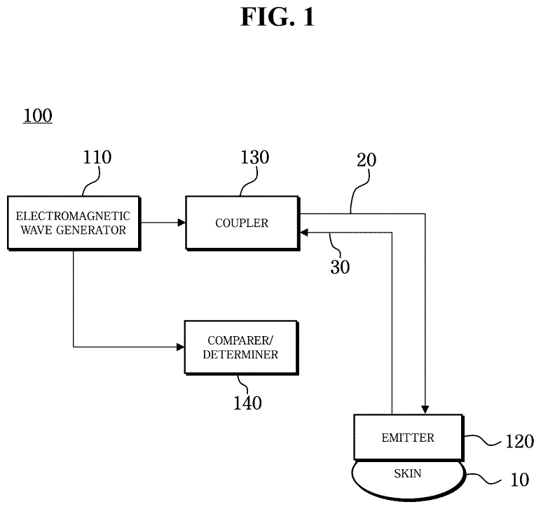

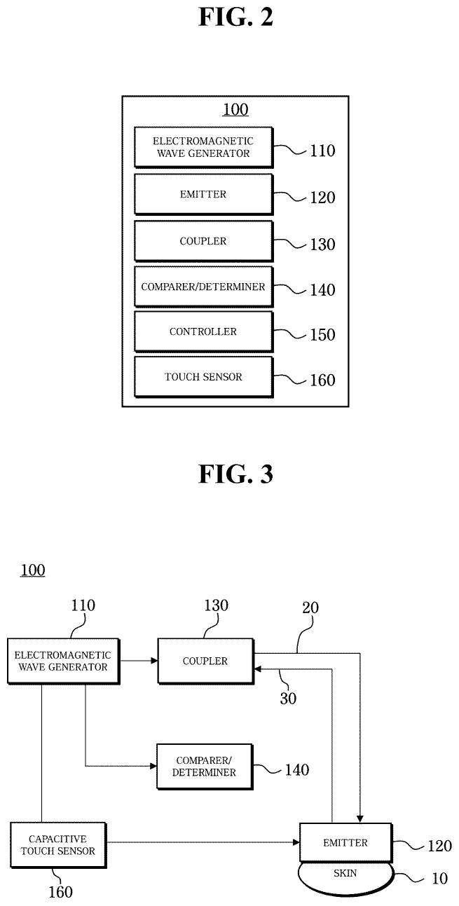

First, FIG. 1 is a configuration diagram illustrating an electromagnetic wave therapy device related to the present disclosure and FIG. 2 is a block diagram of an electromagnetic wave therapy device according to an embodiment of the present disclosure. Hereinafter, an electromagnetic wave therapy device according to a first embodiment of the present disclosure is described with reference to FIG. 1 and FIG. 2.

The electromagnetic wave therapy device 100 according to a first embodiment of the present disclosure includes an electromagnetic wave generator 110 that generates a traveling wave 20, an emitter 120 that receives the traveling wave 20 and emits a reflected wave 30, a coupler 130 that connects the electromagnetic wave generator 110 and the emitter 120 and measures the magnitudes of the traveling wave 20 and the reflected wave 30, a comparer/determiner 140 that operates the electromagnetic wave generator 110 when the ratio of the measured traveling wave 20 and reflected wave 30 reaches a preset value, and a controller 150 that operates the electromagnetic wave generator 110 only when the emitter 120 is in contact with a skin 10 by controlling the electromagnetic wave generator 110, the emitter 120, the coupler 130, and the comparer/determiner 140. The electromagnetic wave therapy device 100 according to an embodiment of the present disclosure treats a body by emitting electromagnetic waves only when the emitter 120 is in contact with a skin 10, as described above.

To this end, it is important to determine whether the emitter 120 is in contact with a skin 10. In an embodiment of the present disclosure, the traveling wave 20 is generated from the electromagnetic wave generator 110 by comparing the magnitudes of the traveling wave 20 and the reflected wave 30 and determining that the emitted wave has come in contact with a skin 10 when the traveling wave 20 is larger than a preset value in comparison to the reflected wave 30.

If the ratio of the traveling wave 20 and the reflected wave 30 is smaller than a preset value, it is required to immediately stop the electromagnetic wave generator 110 and check in more detail whether the reflected wave 30 has come in contact with the skin. Accordingly, in an embodiment of the present disclosure, the electromagnetic wave generator 110 outputs a traveling pulse having minimum electromagnetic wave energy sufficient for detection of a traveling wave 20 and a reflected wave 30, the comparer/determiner 140 measures the ratio of the traveling pulse and a reflected pulse reflected by the emitter 120, and when the ratio of the traveling pulse and the reflected pulse reaches a preset value, it is determined that the skin 10 and the emitter 120 are in contact, and the electromagnetic wave generator 110 is operated. The traveling pulse in this case may be a pulse wave or a low-power electromagnetic wave that has small energy such that the traveling pulse and the reflected pulse can be detected.

Meanwhile, when a detector including a touch sensor 160 is used for the emitter 120 made of metal, it is possible to more easily know whether the emitter 120 is in contact with a human body. The capacitance value generated between the emitter 120 and a human body is the highest when the emitter 120 is in contact with the human body, so, by using this, it is possible to determine that the emitter 120 is in contact with the skin 10 when capacitance over a predetermined level is detected.

By using this, in a second embodiment of the present disclosure, it is possible to determine whether an emitted wave and a skin 10 are in contact using a capacitive touch sensor 160. That is, FIG. 3 is a configuration diagram of an electromagnetic wave therapy device 100 including the capacitive touch sensor 160 according to another embodiment of the present disclosure and it is described hereafter with reference to FIG. 2 and FIG. 3.

According to the second embodiment of the present disclosure, the electromagnetic wave therapy device 100 includes: an electromagnetic wave generator 110 that generates a traveling wave 20; an emitter 120 that receives the traveling wave 20 and emits a reflected wave 30; a capacitive touch sensor 160 that is disposed between the electromagnetic wave generator 110 and the emitter 120 and measures the capacitance between a skin 10 and the emitter 120; a comparer/determiner 140 that operates the electromagnetic wave generator 110 when the value of the capacitance measured by the capacitive touch sensor 160 reaches a preset value, and that stops the electromagnetic wave generator 110 when the value of the capacitance is smaller than the preset value; and a controller 150 that operates the electromagnetic wave generator 110 only when the emitter 120 is in contact with the skin 10 by controlling the electromagnetic wave generator 110, the emitter 120, the capacitive touch sensor 160, and the comparer/determiner 140. In the second embodiment, a coupler 130 connecting the electromagnetic wave generator 110 and the emitter 120 may also be included.

When the capacitance that is measured by the capacitive touch sensor 160 is larger than the preset value, it means that a skin 10 and the emitter 120 are in contact, and when the measured capacitance is smaller than the preset value, it means that a skin 10 and the emitter 120 are not in contact. By applying this, it is possible to operate the electromagnetic wave generator 110 only when a skin 10 and the emitter 120 are in contact.

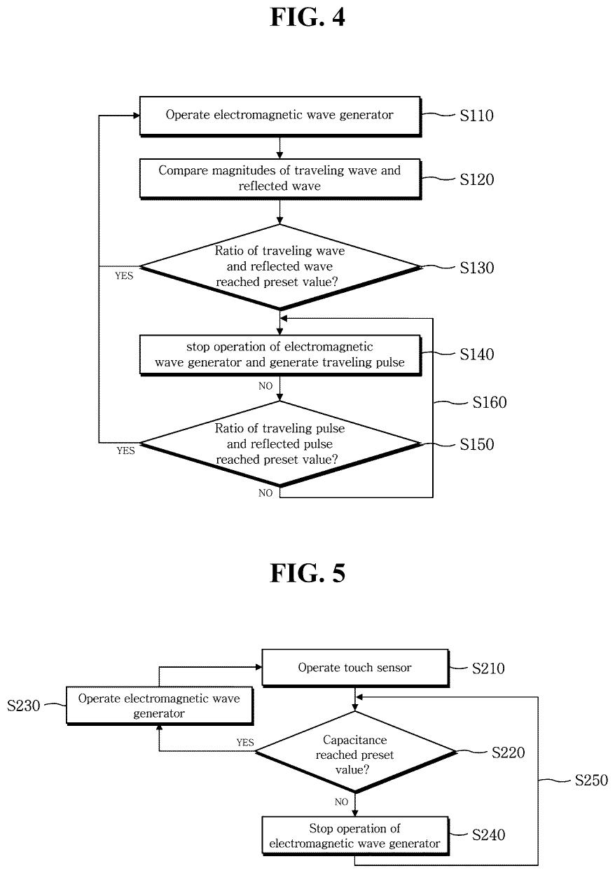

FIG. 4 is a flowchart of a treatment method using the electromagnetic wave therapy device 100 related to the present disclosure and FIG. 5 is a flowchart of a treatment method using the electromagnetic wave therapy device related to the second embodiment. Hereinafter, an electromagnetic wave treatment method is described with reference to FIG. 1 to FIG. 5.

First, an electromagnetic wave treatment method using the electromagnetic wave therapy device 100 that operates the electromagnetic wave generator 110 only when the emitter 120 is in contact with a skin 10 using the configuration of the first embodiment described above is described (see FIG. 1, FIG. 2, and FIG. 4).

The electromagnetic wave treatment method using the electromagnetic wave therapy device 100 includes a step of operating the electromagnetic wave generator 110 (S110), a step of comparing the measured magnitudes of the traveling wave 20 and the reflected wave 30 (S120), and a step of determining whether the ratio of the traveling wave 20 and the reflected wave 30 reaches a preset value (S130). When the ratio of the traveling wave 20 and the reflected wave 30 is larger than the preset value in the determining step S130, the electromagnetic wave generator 110 is operated such that the electromagnetic wave generator 110 is operated only when the emitter 120 is in contact with a skin 10. That is, primarily, when the ratio of the traveling wave 20 and the reflected wave 30 generated from the electromagnetic wave generator 110 reaches the preset value, it is determined that the emitter 120 and a skin 10 are in contact, and the electromagnetic wave generator 110 is operated.

If the ratio of the traveling wave 20 and the reflected wave 30 is smaller than the preset value in the step of determining the magnitudes of the traveling wave 20 and the reflected wave 30 (S130), it is required to determine in more detail whether the emitter wave is in contact with the skin 10. To this end, the method further includes a step of stopping output of the traveling wave 20 and generating a traveling pulse from the electromagnetic wave generator 110 (S140), a step of comparing the magnitudes of the traveling pulse and a reflected pulse from the emitter 120 (S150), and a step of performing the step S110 when the ratio of the traveling pulse and the reflected pulse is larger than a preset value, and performing the step S140 when the ratio of the traveling pulse and the reflected pulse is smaller then the preset value (S160). That is, even though the ratio of the traveling wave 20 and the reflected wave 30 is smaller than the preset value in the step S130, it is determined whether the ratio of fine traveling pulse and reflected pulse reaches the preset value, so that when the ratio of the traveling pulse and reflected pulse reaches the preset value, it is determined that the emitter 120 is in contact with a skin 10 and the electromagnetic wave generator 110 is operated, and when the ratio does not reach the preset value, the traveling pulse is generated and the ratio of the traveling pulse and reflected pulse is repeatedly measured until the ratio of the traveling pulse and reflected pulse reaches the preset value.

As another embodiment, FIG. 5 is a flowchart of a treatment method using the electromagnetic wave therapy device 100 related to the second embodiment.

Hereinafter, an electromagnetic wave treatment method using the electromagnetic wave therapy device 100 that operates the electromagnetic wave generator 110 only when the emitter 120 is in contact with a skin 10 using the configuration of the second embodiment described above is described (see FIG. 2, FIG. 3, and FIG. 5).

The electromagnetic wave treatment method using the capacitive touch sensor 160 includes a step of operating the capacitive touch sensor 160 (S210), a step of comparing capacitance detected by the capacitive touch sensor 160 and a preset value by means of the comparer/determiner 140 (S220), and a step of operating the electromagnetic wave generator 110 when the detected capacitance is larger than the preset value (S230). In this case, when the capacitance reaches the preset value, it is determined that the emitter 120 and a skin 10 are in contact, and the electromagnetic wave generator 110 is operated.

If the capacitance does not reach the preset value, it is determined that the emitter 120 and a skin 10 are not in contact, and operation of the electromagnetic wave generator 110 is stopped (S240), and the step S220 of determining whether the capacitance reaches the preset value is repeatedly performed (S250). This process is repeated until the capacitance reaches the preset value, whereby the electromagnetic wave generator 110 is operated only when the emitter 120 and a skin 10 are in contact.

As described above, in an embodiment of the present disclosure, not only it is possible to check a traveling wave 20 and a reflected wave 30 and determine whether to operate the electromagnetic wave generator 110, depending on the ratio of the traveling wave 20 and the reflected wave 30, but it is possible to selectively control operation of the electromagnetic wave generator 110, depending on the capacitance value measured by the capacitive touch sensor 122. That is, the capacitive touch sensor 122 is operated with preset power in at least one of the case when contact with a skin 10 is determined or the case when the ratio of a traveling wave 20 and a reflected wave 30 reaches a preset minimum value. Alternatively, it is also possible to perform control using both of the two methods.

In this case, when the capacitive touch sensor is used, whether there is contact is determined using the capacitive touch sensor 160 through the controller 150 and operation of the electromagnetic wave generator 110 is controlled.

The detailed description should not be construed as being limited in all respects and should be construed as an example. The scope of the present disclosure should be determined by reasonable analysis of the claims and all changes within an equivalent range of the present disclosure are included in the scope of the present disclosure.

What is claimed is:

1. An electromagnetic wave therapy device that treats a skin by generating an electromagnetic wave at an end that is in contact with a skin, the electromagnetic wave therapy device comprising:
an electromagnetic wave generator generating a traveling wave;
an emitter receiving the traveling wave and emitting a reflected wave;
a coupler connecting the electromagnetic wave generator and the emitter and measuring magnitudes of the traveling wave and the reflected wave;
a comparer/determiner operating the electromagnetic wave generator when a ratio of the measured traveling wave and reflected wave reaches a preset value; and
a controller operating the electromagnetic wave generator only when the emitter is in contact with the skin by controlling the electromagnetic wave generator, the emitter, the coupler, and the comparer/determiner.

2. The electromagnetic wave therapy device of claim 1, wherein when the ratio of the traveling wave and the reflected wave is smaller than the preset value, the electromagnetic wave generator outputs a traveling pulse sufficient for detection of a traveling wave and a reflected wave, and the comparer/determiner measures a ratio of the traveling pulse and a reflected pulse reflected by the emitter, and
when the ratio of the traveling pulse and the reflected pulse reaches a preset value, the electromagnetic wave generator is operated.

3. The electromagnetic wave therapy device of claim 2, wherein the traveling pulse is a pulse wave or a low-power electromagnetic wave that has electromagnetic wave energy sufficient for detection of the traveling pulse and the reflected pulse.

4. An electromagnetic wave therapy device that treats a skin by generating an electromagnetic wave at an end that is in contact with a skin, the electromagnetic wave therapy device comprising:
an electromagnetic wave generator generating a traveling wave;
an emitter receiving the traveling wave and emitting a reflected wave;
a capacitive touch sensor disposed between the electromagnetic wave generator and the emitter and measuring capacitance between the skin and the emitter;
a comparer/determiner operating the electromagnetic wave generator when the value of the capacitance measured by the capacitive touch sensor reaches a preset value, and stopping the electromagnetic wave generator when the value of the capacitance is smaller than the preset value; and
a controller operating the electromagnetic wave generator only when the emitter is in contact with the skin by controlling the electromagnetic wave generator, the emitter, the capacitive touch sensor, and the comparer/determiner.

5. An electromagnetic wave treatment method using an electromagnetic wave therapy device that includes an electromagnetic wave generator generating a traveling wave, an emitter receiving the traveling wave and emitting a reflected wave, a coupler connecting the electromagnetic wave generator and the emitter and measuring magnitudes of the traveling wave and the reflected wave output from the electromagnetic wave generator, a comparer/determiner operating the electromagnetic wave generator when a ratio of the measured traveling wave and reflected wave reaches a preset value, and a controller operating the electromagnetic wave generator only when the emitter is in contact with the skin by controlling the electromagnetic wave generator, the emitter, the coupler, and the comparer/determiner, the electromagnetic wave treatment method comprising:
(a) operating the electromagnetic wave generator;
(b) comparing the measured magnitudes of the traveling wave and the reflected wave; and
(c) determining whether a ratio of the traveling wave and the reflected wave reaches a preset value,
wherein when the ratio of the traveling wave and the reflected wave is larger than the preset value in the determining, the electromagnetic wave generator is operated such that the electromagnetic wave generator is operated only when the emitter is in contact with the skin.

6. The electromagnetic wave treatment method of claim 5, further comprising:
when the ratio of the traveling wave and the reflected wave is smaller than the preset value in the comparing of the magnitudes of the traveling wave and the reflected wave,
(d) stopping output of the traveling wave and generating a traveling pulse from the electromagnetic wave generator;
(e) comparing magnitudes of the traveling pulse and a reflected pulse from the emitter; and
(f) performing the step (a) when a ratio of the traveling pulse and the reflected pulse is larger than a preset value, and performing the step (d) when the ratio of the traveling pulse and the reflected pulse is smaller than the preset value.

7. An electromagnetic wave treatment method using an electromagnetic wave therapy device that includes an electromagnetic wave generator generating a traveling wave, an emitter receiving the traveling wave and emitting a reflected wave, a coupler connecting the electromagnetic wave generator and the emitter and measuring magnitudes of the traveling wave and the reflected wave, a capacitive touch sensor connected at both ends to the emitter and the electromagnetic wave generator, respectively, and detecting capacitance when the emitter is in contact with) a skin, a comparer/determiner operating the electromagnetic wave generator when capacitance detected by the capacitive touch sensor is larger than a preset value, and a controller operating the electromagnetic wave generator only when the emitter is in contact with the skin by controlling the electromagnetic wave generator, the emitter, the coupler, the capacitive touch sensor, and the comparer/determiner, the electromagnetic wave treatment method comprising:
(g) operating the capacitive touch sensor;
(h) comparing capacitance detected by the capacitive touch sensor and a preset value by means of the comparer/determiner; and
(i) operating the electromagnetic wave generator when the detected capacitance is larger than the preset value.

8. The electromagnetic wave treatment method of claim 7, wherein when the detected capacitance is smaller than the preset value in the step (h), output of the traveling wave from the electromagnetic wave generator is stopped and the step (h) is performed.
